(19) United States
(12) Patent Application Publication
(10) Pub. No.: US 2006/0084848 A1
(43) Pub. Date: Apr. 20, 2006

(54) Apparatus and methods for monitoring subjects

(76) Inventor: Mark Mitchnick, East Hampton, NY (US)

Correspondence Address: Dwight H. Renfrew, Esq.;Ohlandt, Greeley, Ruggiero & Perle, L.L.P., 10th Floor, One Landmark Square, Stamford, CT, 06901-2682, US

(21) Appl. No.: 10/965,010

(22) Filed: Oct. 14, 2004

(51) Int. Cl. A61B 5/00; A61B 5/103

(52) U.S. Cl. 600/301; 600/549; 128/903; 600/595; 600/591; 600/573

(57) ABSTRACT

This invention provides a device, and method and system for its use, for monitoring participants in clinical trials so that participant self-reporting, which is known to be notoriously inaccurate, can be minimized or eliminated. In preferred embodiments, the device is self-contained and self-powered, resides on or in a body cavity of the participant, collects data monitoring medically relevant aspects of the participant's behavior and of the local device environment, and stores data in a memory on-board the device. An accompanying external station reads stored data and prepares it for use. Devices may include electrically-active sensors and non-electrical active sampling sensors. A preferred embodiment of the device is in clinical trials of microbicides inhibiting transmission of the HIV virus.

### 1. FIELD OF THE INVENTION

The present invention, in a preferred embodiment, relates to monitoring participants in clinical trials of pharmaceuticals relating to sexual activity, especially those for limiting or preventing transmission of sexually transmitted diseases (STDs), and provides a device for participant monitoring with accompanying systems and methods that automatically collects monitoring data with little or no participant attention.

### 2. BACKGROUND OF THE INVENTION

Women now account for the majority of people with human immunodeficiency virus (HIV) infections, and young women bear the brunt of new infections in many parts of the world. See Copan et al., 2004, Science 304:1911 (and the references contained therein). Advances in understanding the molecular mechanisms of HIV sexual transmission are focusing new interest and resources on development of topical intra-vaginal agents that block HIV infection. Even if these agents, known as microbicides, are only partially effective at preventing infection, they will hugely complement existing prevention and treatment methods. Mathematical modeling studies estimate that a partially effective microbicide used in half of coital acts by 20% of women at risk could prevent 2.5 million infections in 3 years.

Many new microbicides are now under development. More than 40 new compounds are being tested in the laboratory; many new compounds are in clinical testing; and six cand dates are currently in or about to enter phase III studies. Further, new microbicide preparations containing two or more active ingredients, which are likely to be more effective than single active agent preparations, will require independent laboratory and clinical testing.

Yet there are significant and unique obstacles to microbicide testing and licensing. Chronic use of topical intravaginal drugs to prevent infection is a new concept with which regulators have little previous experience. There is no surrogate marker or animal model known to reliably predict microbicide efficacy in humans. Since microbicides are expected to be effective only on average, trials require larger numbers of participants. All these factors suggest that large clinical trials are, if anything, even more necessary to demonstrate the safety and efficacy of microbicides.

But the logistics and costs of these studies are formidable. Intrinsic characteristics of microbicides require that trials have up to tens of thousands of HIV-free, but high-risk women participants and that the participants be followed for up to several years. During licensing delays required by this testing, large numbers of new infections will unnecessarily occur if new drugs prove efficacious. Further, many trials will necessarily involve participants in developing countries subject to economic, environmental and social stresses. Finally, against this complex background, trial data must currently rely essentially on self-reporting of sexual habits, which is known to be notoriously inaccurate even in the best conditions.

Thus, it is a worldwide priority to expand capacity for rapidly, cheaply, and easily performing efficacy trials of microbicides and other preventions. Targeted efforts and significant financial investment by several organizations have established clinical sites capable of conducting these microbicide trials according to international guidelines. However, these sites are overburdened already with the current candidates for phase III trials.

Therefore, systems and methods that help make such trials simpler and more economical are urgently needed and will have great benefit. For example, systems and methods providing more accurate and up-to-date trial data can reduce the number of participants required and shorten the time to demonstrate (or not) the efficacy of candidate drugs. Such systems and methods are not currently available.

A number of references are cited herein, the entire disclosures of which are incorporated herein, in their entirety, by reference for all purposes. Further, none of these references, regardless of how characterized above, is admitted as prior to the invention of the subject matter claimed herein.

### 3. SUMMARY OF THE INVENTION

The objects of the present invention are to provide unobtrusive devices for automatically monitoring participants in clinical trials of topical pharmaceutical agents for limiting or preventing STD transmission, and also in trials of other types of pharmaceutical agents, especially pharmaceutical agents relating to sexual activity. For example, because anti-depressants and similar psychoactive agents are known to affect patient-reported libido, it may be advantageous to objectively monitor patients taking such agents. These objects also include providing systems and methods for use of such monitoring devices in clinical trials.

Further objects of the present invention include monitoring subjects whether or not engaged in clinical trials. Preferred devices reside in a body cavity and can provide access to body-core values for, for example, temperature, glucose, pO2, and the like. Also, the present invention can monitor menstrual cycles and their characteristics. Additionally, the present invention can be combined with drug delivery devices similarly configured to reside in a body cavity, and thereby provide passive or actively-triggered drug delivery.

The inventive principles to be described herein provide for monitoring devices that are designed to be affixed to, or to reside in a cavity of, a participant in a clinical trial (or otherwise the subject of a study) with little or no discomfort or risk for extended periods, e.g., weeks, a month, or several months, or up to a year or more. These devices incorporate sensors and supporting components for sensing and recording data relevant to the clinical trial or study. This data advantageously includes participant behavioral data that would otherwise require participant self-reporting or invasive devices. Sensors usually detect the local environment where a topical pharmaceutical is to be applied, whether or not the topical pharmaceutical has been applied, and the like. This data is stored, at least temporarily, in the device, and later off loaded and processed to provide useful participant monitoring data. The preferred practical application of these principles, and the preferred but not limiting embodiments of the device, is as an intra-vaginal device used during trials of pharmaceuticals, especially microbicides, for preventing or limiting STD (e.g., HIV) transmission. The following description is directed and exemplified largely in terms of these preferred but non-limiting embodiments.

Accordingly, a preferred intra-vaginal device of this invention includes sensors for, at least, detecting and storing the occurrences of sexual activity (primarily, intercourse). Sexual activity is preferably detected by observing characteristic patterns of participant motion as sensed by an acceleration. Preferably, devices also include sensors for other aspects of the vaginal environment and for detecting the application or presence of topical pharmaceuticals. Additional sensors that can be part of a device include temperature sensors, pH sensors, heart rate sensors, pO2 sensors (based on pulse oximeter electronics), and the like. If available, sensors for specific chemicals can be included, such as sensors for selected pharmaceuticals, microbicides, spermicides, and the like. Further, chemical or physical labels can be added to pharmaceuticals, pharmaceutical applicators, and the like, to simplify their detection. One preferred physical label is magnetic micro-beads or other magnetic materials in combination with a compact magnetic field sensor in a device.

A preferred device also includes supporting components for making use of these electrically-active types of sensors. Preferably, a device includes a micro-controller (MC), or the equivalent, for retrieving data from sensors, for storing retrieved data in an on-board memory, and for controlling overall device operation, especially by managing power use for longer battery life. A device further advantageously includes data memory, power management circuitry, and other components known in the art. Data compression is preferably used to conserve memory. All components are selected to have compact form factors and low and/or controllable power consumption.

In one preferred embodiment, these sensors and supporting components are packaged into a single unit sized and shaped for residing intra-vaginally in a monitored participant. It is paramount that a device be safe, convenient, comfortable, and acceptable to participants, and that it interfere only minimally, or not at all, with the participants normal sexual activities. A preferred configuration is ring-shaped and sized to reliably reside in the back of the vagina adjacent to the cervix much like a diaphragm or cervical cap. Ring-shaped intra-vaginal devices have been used for drug delivery and other applications, and patients have found them comfortable and acceptable. See, for example, U.S. Pat. No. 4,827,946, U.S. Pat. No. 5,928,195, and Rathbone et al. eds., 2003, Modified-Release Drug Delivery Technology, C.H.I.P.S., Weimar, Tex. (all presenting further details of intra-vaginal ring technology; and all incorporated by reference herein in their entireties for all purposes). Alternate physical configurations, such a cylindrical shape, are also within the scope of this invention. Devices are preferably constructed from medical-grade silicone elastomers, as these materials have already been proven suitable in intra-vaginal drug-delivery rings.

In other preferred embodiments, the device may be configured into two (or more) separate units. In one such configuration, the device includes an intra-vaginal unit and a cooperating extra-vaginal unit, the extra-vaginal unit being on or near the participant, for example, being supported on the participant's clothing. These two units communicate wirelessly using one of the available very low power, short range radio link protocols (e.g., Bluetooth) now available as single chip integrated circuit (IC) transceivers. For example, the intra-vaginal unit can retrieve sensor data and transmit it to the extra-vaginal unit; the extra-vaginal unit can receive and store sensor data and perform overall management of both units. The intra-vaginal can then use simpler, smaller, and more power efficient supporting components, while the larger components requiring more power, such as the MC, may reside externally where the physical constraints are less. Alternatively, the extra-vaginal unit may be supplemented or replaced by an external station to which data is be transmitted from time-to-time to free memory in the intra-vaginal unit. In a further alternative, the two units may include an inductive coupling (e.g., forming a transformer) so that the extra-vaginal unit can recharge from time-to-time batteries in the intra-vaginal unit. It is then possible, for example, that the intra-vaginal unit be even more compact by dispensing with batteries and power control. The extra-vaginal unit then provides power to the intra-vaginal unit only when needed to retrieve and transmit sensor data; otherwise, the intra-vaginal unit is inactive. Finally, in other embodiments, the intra-vaginal unit may be entirely dispensed with, and all data sensed from an external unit on or near the participant.

In the following, without limitation, the device of this invention is described largely in its single-unit embodiment. Constructing an embodiment with two (or more) units simply requires, first, that the functions and components of the single unit be distributed among the two units, and second, that a wireless link component and/or optional inductive coupling by added to both units. Wireless links and inductive coupling are already known and used in the art. Therefore, in view of the following description of the single-unit embodiments, it will be readily apparent to one of ordinary skill in the art how other embodiments can be constructed.

Finally, this invention includes also systems and methods for making device monitoring data available for its ultimate uses. Data is retrieved or read-out from the single unit device usually when the device is removed from the participant. Preferably, electrical contact pads provided on the device interface to an external data reader so that data can non-destructively be moved from the on-board memory to an external station or system, such as a standard personal computer (PC). (In multi-unit embodiments, data can be wirelessly retrieved from time-to-time without device removal.) The external station performs basic processing, such as extracting data from the format in which it was stored in device on-board memory and correcting apparent sensor errors (such as baseline drift). After such processing, the data is transmitted or sent for its ultimate uses.

A device can also advantageously include electrically-non-active sensors. Preferred electrically-non-active sensors include passive physical sensors designed to sample and retain components encountered in the device's (intra-vaginal) environment. Components sampled by such a sensor are detected upon device removal using known chemical and biological techniques that are usually destructive of the sensor. A preferred embodiment of a sampling sensor includes an absorbent material disposed in an enclosed cavity in the device, the cavity communicating with the external device environment through pores and/or a membrane. Characteristics of the membrane and/or absorbent material can be selected to give such a sampling sensor a degree of selectivity and specificity. For example, correctly sized membrane pore can exclude external components larger that desired; or surface treatment of membrane pores can favor the passage of desired classes of external components. The absorbent material can be selected to preferentially retain desired external components, such as compounds, biologicals, or biological agents, by being hydrophobic, or hydrophilic, or positively charged, or negatively charged, or neutral, or the like. Further, the absorbent material can be selected to selectively bind and retain selected external components. For example, this material can have bound antibodies specific for viruses, such as hepatitis C, HPV, HIV, and the like, and other biological agents. In another example a sensor specific for PSA (prostate specific antigen) can be present. PSA, being only present in males, would be indicative of unprotected intercourse.

In a first embodiment, this invention includes an apparatus for monitoring a subject which comprises a housing adapted to reside in an anatomic cavity of the subject's body, the housing having no external physical connection, and at least one sensor of acceleration within the housing.

Aspects of the first embodiment further include: that the anatomic cavity normally opens externally; that the housing is further adapted to permit insertion and removal by the subject; that the anatomic cavity is a vagina; that the housing is ring-shaped; that at least one sensor is selected from the group consisting of a sensor of pH, a sensor of temperature, a sensor of the occurrence of menstruation, a sensor of the proximity of magnetic materials, a sensor of the proximity of a target object.

Additional aspects of the first embodiment include: a computer-readable memory within the housing, and a controller within the housing that retrieves measurements from at least one sensor and stores the retrieved measurements in the memory; or an external unit, and an RF transmitter within the housing that transmits sensor measurements to the external unit; or a sampling sensor that samples components present in the external environment of the apparatus that comprises an absorbent material disposed in a cavity in the housing, and one or more pores through which components of the external environment have access to the absorbent material, wherein components are sampled by being absorbed by the absorbent material after passing though the pores from the external environment; or a biosensor for sensing biological components that is disposed in the housing and having access to biological components present in the external environment.

Additional aspects of the first embodiment include: that the biosensor comprises antibodies selective for one or more biological components; that the antibodies are selective for one or more sexually transmitted disease agents; that sexually transmitted disease agents comprise the human immunodeficiency virus (HIV).

In a second embodiment, this invention includes an apparatus for monitoring a female subject that comprises a housing adapted to reside in the subject's vagina, the housing having no external physical connection, and one or more sensors within the housing comprising at least one sensor selected from the group consisting of a sensor of acceleration, a sensor of pH, a sensor of temperature, a sensor of the occurrence of menstruation, a sensor of the proximity of magnetic materials, and a sensor of the proximity of a target object.

Aspects of the second embodiment further include: that peak values of the measured acceleration and the occurrence times of the measured peak values are determined; that the subject is determined as likely to be engaging in sexual activity, or as not likely to be engaging in sexual activity, by comparing characteristics of the acceleration peak values and of the peak-value occurrence times with characteristics expected if the subject is actually engaging in a sexual activity.

In a third embodiment, this invention includes an apparatus for monitoring a subject that comprises a housing adapted to reside in an anatomic cavity of the subject's body, the housing having no external physical connection, and at least one sensor of the proximity of magnetic materials within the housing. The third embodiment is advantageously used with a pharmaceutical preparation applied in the proximity of the apparatus that includes magnetic materials.

In a fourth embodiment, this invention includes an apparatus for monitoring a subject that comprises a housing adapted to reside in an anatomic cavity of the subject's body, the housing having no external physical connection, and at least one sensor of the proximity of a target object within the housing. Aspects of the fourth embodiment further include: that the proximity sensor generates an electromagnetic field and senses perturbations in the electromagnetic field, and wherein the target object perturbs an electromagnetic field and is thereby detected; that the target object comprises a conductive element and/or an inductive element that perturbs an electromagnetic field; that the target object comprises an applicator for a pharmaceutical; that the target object comprises a contraceptive device; or that the contraceptive device comprises a condom.

In a fifth embodiment, this invention includes an apparatus for monitoring a subject that comprises a housing adapted to reside in an anatomic cavity of the subject's body, the housing having no external physical connection, and a sampling sensor for sampling components of the external environment of the apparatus, the sampling sensor comprising an absorbent material disposed in a cavity in the housing, and one or more pores through which components of the external environment have access to the absorbent material, wherein components are sampled by being absorbed by the absorbent material after passing though the pores from the external environment.

In a sixth embodiment, this invention includes an apparatus for monitoring a female subject's sexual activity that comprises a housing adapted to reside in the subject's vagina, the housing having no external physical connection, one or more sensors within the housing, a computer-readable memory within the housing, a controller within the housing, and a program for awakening periodically from a low power sleep state in order to determine from sensor measurements whether or not the subject is likely to be engaging in sexual activity, retrieving measurements from at least one sensor and storing the retrieved measurements in the memory during a period in which the subject is determined as likely to be engaging in sexual activity, and switching back to the low power sleep state if the subject is determined as not likely to be engaging in sexual activity. An aspect of the sixth embodiment includes that acceleration measurements are adaptively retrieved and stored so that the acceleration is more frequently sampled near the expected times of an acceleration peak value, and is less frequently sampled between the expected times of acceleration peak values.

In a seventh embodiment, this invention includes an apparatus for monitoring and drug delivery that comprises a housing adapted to reside in an anatomic cavity of the subject's body, the housing having no external physical connection; the housing comprising an embedded pharmaceutical agent that diffuses from the housing into the anatomic cavity, and one or more sensors within the housing. Aspects of the seventh embodiment further include: that the pharmaceutical agent is embedded throughout the housing material; or that the pharmaceutical agent is embedded in a core of the housing.

In an eighth embodiment, this invention includes an apparatus for subject monitoring and drug delivery that comprises a housing adapted to reside in an anatomic cavity of the subject's body, the housing having no external physical connection, a reservoir of a pharmaceutical agent within the housing, and one or more sensors within the housing, a controller within the housing, and a program for retrieving measurements from at least one sensor and determining whether or not a condition is satisfied in dependence on the retrieved measurements, and if the condition is determined to be satisfied, signaling that an amount of the pharmaceutical agent is to be released in the anatomic cavity. An aspect of the eighth embodiment further includes that the condition to be satisfied comprises whether the subject is likely to be engaging in a sexual activity.

In a ninth embodiment, this invention includes a computer readable memory with a program for performing the methods of this invention. An aspect of the ninth embodiment further comprises that the method comprises awakening periodically from a low power sleep state in order to determine from acceleration measurements whether or not the subject is likely to be engaging in sexual activity, retrieving measurements from at least one sensor and storing the retrieved measurements in the memory during a period in which the subject is determined as likely to be engaging in sexual activity, and switching back to the low power sleep state if the subject is determined as not likely to be engaging in sexual activity.

In a tenth embodiment, this invention includes a system from monitoring female subjects that comprises a monitoring apparatus comprising a housing adapted to reside in the subject's vagina, the housing having no external physical connection, and one or more sensors within the housing comprising at least one sensor selected from the group consisting of a sensor of acceleration, a sensor of pH, a sensor of temperature, a sensor of the occurrence of menstruation, a sensor of the proximity of magnetic materials, and a sensor of the proximity of a target object, a computer-readable memory within the housing, and a controller within the housing that retrieves measurements from at least one sensor and stores the retrieved measurements in the memory, and a computer for reading measurements stored in the memory of the monitoring apparatus during a prior period of residence in the subject.

In an eleventh embodiment, this invention includes a method of conducting clinical trials using a plurality of subjects that comprises providing to at least one subject a monitoring apparatus comprising a housing adapted to reside in the subject's vagina, the housing having no external physical connection, and one or more sensors within the housing comprising at least one sensor selected from the group consisting of a sensor of acceleration, a sensor of pH, a sensor of temperature, a sensor of the occurrence of menstruation, a sensor of the proximity of magnetic materials, and a sensor of the proximity of a target object, a computer-readable memory within the housing, and a controller within the housing that retrieves measurements from at least one sensor and stores the retrieved measurements in the memory, reading measurements stored in the memory of the monitoring apparatus after a period of subject use, and transmitting the measurements read for analysis.

In a twelfth embodiment, this invention includes a pharmaceutical preparation that comprises one or more pharmaceutical agents; and embedded magnetic materials sufficient to permit a sensor for magnetic materials to sense the proximity of the pharmaceutical preparation.

In an thirteenth embodiment, this invention includes an apparatus for monitoring a female subject's sexual activity that comprises a housing adapted to reside in the subject's vagina, the housing having no external physical connection, one or more sensors including at least one sensor of acceleration within the housing, a controller within the housing that determines whether or not the subject is likely to be engaging in sexual activity, or is not likely to be engaging in sexual activity, by comparing characteristics of the acceleration signals with characteristics expected if the subject is actually engaging in a sexual activity.

Further this invention includes combinations and subcombinations of the various embodiments, and aspects described herein.

### 4. BRIEF DESCRIPTION OF THE DRAWINGS

The present invention may be understood more fully by reference to the following detailed description of preferred embodiments of the present invention, illustrative examples of specific embodiments of the invention, and the appended figures in which:

FIG. 1 illustrates an exemplary of the device;

FIG. 2 illustrates a block diagram of the device;

FIG. 3 illustrates on-line device processing;

FIGS. 4A-B illustrate off-line processing of device data; and

FIGS. 5A-C illustrate an example of an embodiment of this invention.

### 5. DETAILED DESCRIPTION OF THE PREFERRED EMBODIMENTS

The preferred embodiments of this invention are single-unit, intra-vaginal monitoring devices that include sensors for detecting sexual activity, and optionally, also for detecting/sampling other aspects of the intra-vaginal environment. This invention also includes multi-unit embodiments, and further embodiments for residing elsewhere in or on the body and detecting other parameters of medical/clinical interest. Headings are used here, and throughout this application, for clarity and convenience only.

Device Configuration and Components

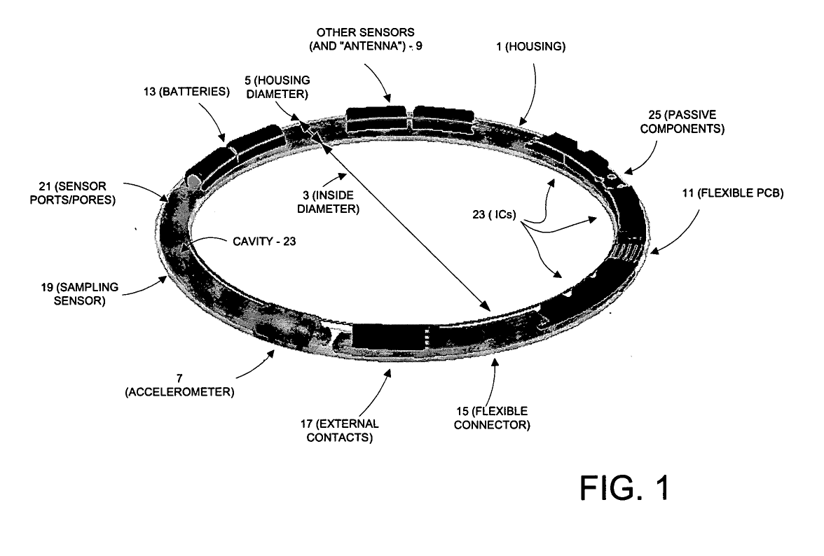

With reference to FIG. 1, device housing 1, which encloses the device's sensors and supporting components, is shaped and sized for safety and subject acceptability, providing, for example, comfort, ease of insertion and removal, convenience, and little or no interference with a participant's (wearer's) sexual activities. A ring shape (torus) with a generally circular to oval cross section to be worn or reside at the top of the vagina about the cervix is preferred. This shape and placement has been accepted by patients for intra-vaginal drug delivery. Alternatively, the device may have one of the other shapes (e.g., cylindrical) and placements accepted for intra-vaginal devices. In typical sizes, the device has inside diameter 3 of from approximately 40 mm to approximately 80 mm and cross section 5 of from approximately 15 mm to approximately 25 mm. Preferably, a range of sizes and/or shapes satisfy particular needs of individual participants.

The device can be conveniently constructed by molding the housing. For example, the housing may be molded in a single step about the internal components after they have been arranged into their designed configuration. Also, a first half of the housing may be injection molded; the internal components arranged therein; and finally second half may then be injection molded thus completing the housing. Alternatively, in a multi-step process, the housing may be molded with a central tunnel or cavity, the internal components arranged into this cavity, and then the housing sealed. The housing's external surface is preferably smooth and unbroken except for optional ports or pores 21 which allow optional sampling sensors to communicate with the device environment. Also electrical contact pads 17 can be disposed near but within the housing surface so that external electrical connection may be easily made with the device in order to, e.g., read out stored data. External connection can be made by external conductive pins (not illustrated) that penetrate the thin housing layer overlying the contact pads.

A suitable housing material should be medically approved/appropriate for internal use, and preferably specifically approved/appropriate for intra-vaginal use. Physically, it should be sufficiently flexible so that it can be deformed for easy insertion, and so that once inserted, it can adapt to the participant; it should be sufficiently rigid to remain reliably in place once inserted; and it should be at least impermeable and non-conductive to protect internal components. Preferred materials include approved (by the U.S. Food and Drug Administration (FDA)) medical grade silicone elastomers including, e.g., poly-siloxanes, particularly poly-dimethyl-siloxanes, copolymers of, for example, dimethyl-siloxanes and methyl-vinyl-siloxanes, and polymers include siloxane derivatives (containing, for example, fluoro- or phenyl-groups). Such materials are available from the Dow Corning (Midland, Mich.). Other suitable elastomers include medical grade formulations of polyurethanes, ethylene/vinyl acetate copolymers, and the like. Rigidity and other physical characteristics of the housing can be controlled and/or enhanced by adding a particulate material such as fumed silica or diatomaceous earth. Surface treatments that increase lubricity and/or decrease allergic potential can improve tolerance and safety.

Sensors present in a particular embodiment reside in, and are protected by, the housing. Certain sensors (“electrically-active” or “electrical” sensors) that detect electrical, and/or magnetic, and/or mechanical signals do not access the device's external environment, although they do require supporting components to retrieve and store their detected data. These sensors are described next, beginning with exemplary supporting components. Certain other sensors (“sampling” sensors) that physically sample the device environment do require external access, although they are generally passive and require no supporting components in the device. They are described subsequently.

Electrically-active sensors and components are sized and arranged so that they can be molded/mounted into ring-shaped housings of the sizes already described without impairing device flexibility. Active circuit components, especially integrated circuits (IC), must be carefully selected to be preferably less than approximately 5-7 mm in all dimensions, and less preferably up to approximately 10-15 mm at most one dimension. It will be appreciated from the following that appropriate ICs are available in the art. Passive components present little selection problem as they are routinely available in sub-millimeter form factors (size 0201-0.06 mm by 0.03 mm—is preferred). ICs and other circuit components are mounted on one (or more than one) flexible printed circuit board (PCB) using a mounting technology suitable for miniaturization, with ball-grid array (BGA) or flip-chip formats generally preferred. The flexible PCB is generally shaped as a portion of an annulus in order to fit within the housing. Construction of variously shaped flexible PCBs are well known in the art.

FIG. 1 illustrates an exemplary physical arrangement of a device's internal components. Supporting active components, component ICs 23, and passive components, components 25, are mounted on flexible PCB 11. By means of extension of separate flexible connector 15, PCB 11 links to support for external contact pads 17. Pads 17 are positioned near the surface of housing 1 for easy external access. Certain electrical/electronic sensors, including accelerometer 7 and other sensors 9, are illustrated as being discretely and separately mounted in the housing; alternatively they may be mounted on a PCB, such as PCB 11. Generally, sensor mounting reflects positional requirements: an accelerometer is mounted so that its acceleration measurements are correctly oriented with respect to the housing; a thermistor is correctly placed near the surface of the housing, and so forth. The illustrated device also includes sampling sensor 19, which is non-electrically active. One or more batteries 13 supplying electrical power are discretely and separately mounted in the housing. Other embodiments of the device may also include an RF transceiver IC for communicating externally and an inductor for inductively coupling to an external power source.

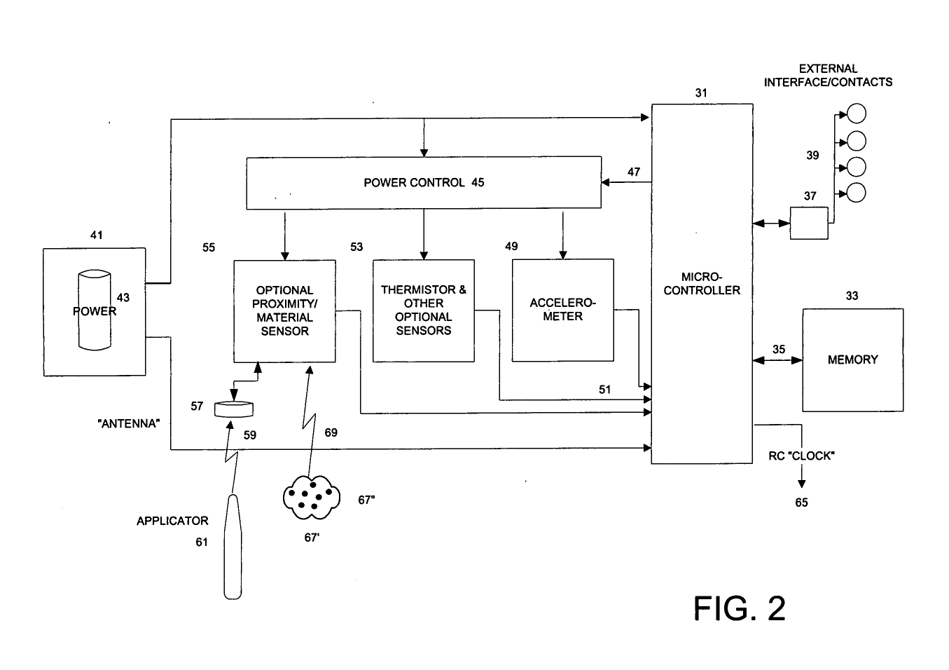

FIG. 2 illustrates an exemplary electrical arrangement of electrically-active sensors and their supporting components. The device is preferably controlled by a highly integrated, low-voltage, low-power micro-controller (MC) 31 in order to minimize device count, size, and power consumption. A preferred MC integrates together a micro-processor which implements general-purpose and power management instructions; program memory, for example flash ROM; temporary data memory, for example, RAM; serial interfaces to, for example, external memory; and also on-chip peripherals for interfacing and processing analog sensor signals. In more detail, power management preferably provides a very low power (significantly less than 1 micro ampere) sleep mode, entered, for example, upon executing a SLEEP instruction, and exited upon internal timer or external interrupts. Optionally, the MC has at least one, selectable, low frequency, and low power mode in addition to a normal frequency normal operational mode. Serial interfaces preferably include at least one universal synchronous/asynchronous interfaces (USART) or the equivalent for known serial communication interfaces such as SCI. I2C, SPI, and the like. Peripherals for analog interfacing include one or more A/D converters, signal comparators, timers, control I/O ports, and the like. These peripherals can particularly reduce external part numbers. Suitable MC parts include the MSP430x13x, MSP430x, 14x, and MSP430x14x1 series micro-controllers from Texas Instruments, Inc. and the PIC 16F627A/628A/648A devices from Microchip Technology, Inc.

The device MC is preferably clocked by compact resistor-capacitor (RC) circuit 65, instead of by a relatively larger crystal. RC circuits have suitable stability for the present device. Any inaccuracies in absolute frequency can be corrected by the MC control software, which may incorporate calibrations determined from an actual frequency measured during device construction. The MC stores retrieved monitoring data into memory 33, which is preferably directly linked to the MC through a serial port 35. Serial EEPROM, 64 kb, or 128 kb, or more, is preferred, and suitable memory parts include Atmel Corp. AT24C128 and AT24C256 2-wire serial EEPROMS (sized less than 4 mm in all dimensions for BGA mounting). A second serial link can provide an external interface 39 through optional driver 37. FIG. 2 illustrates a four-pad external interface 39 (two power and two signal pads). Additional analog parts, for example, scaling and/or sample-and-hold operational amplifiers, beyond those integrated with the MC may be needed in certain embodiments. Suitable parts include LMV301MG or LMV981MG available from National Semiconductor, Inc, in SC-70-5 or -6 packages or in die format.

Extended battery life (for example, a month on longer) is important for the preferred intra-vaginal embodiments. Suitable battery capacity is selected according to the desired device life and expected power load, which varies at least with the number of optional sensors provided in an embodiment. Preferred batteries are, first of all, safe and approved for external medical use; are no more than 10 mm in diameter and 10 mm in length; and have a capacity of from approximately 3 mAh to approximately 30 mAh or more. Suitable batteries include hearing-aid type batteries. Such batteries are widely available in sizes from 6 mm to 8 mm in diameter and from 3 mm to 8 mm in length and with capacities up to 30 mAh or more. Since such batteries are zinc-air, the housing must provide porosity in the region of the battery for the small amount of air required. Also, an air reservoir or pocket may be present the housing in place of, or supplementing, housing porosity. Smaller custom batteries are more preferred. For example, custom batteries down to 3 mm in diameter, 7 mm in length, and with a 3 mAh life and available from Quallion LLC. Power supply 41 may optionally include a voltage regulator to control battery 43's raw supply voltage to a safe level for IC operation. Several appropriate regulator parts are available in BGA or in die form.

The device also preferably includes external power management circuitry 45 controlled by the MC so that sensors and supporting components not in current use can be powered down. When the MC is in sleep mode, all external components not required for MC wakeup are preferably powered down. Power management 45 includes at least one FET switch between the power supply and the external components, and preferably includes one FET switch for each group of separately powered components. The FET switches can be directly controlled by the MC I/O pins. Note that although FIG. 2 illustrates that external components 35, 37, and 39 are not controlled by power management circuit 45, this is for simplicity and clarity only. Most preferred embodiments permit that any external component not necessary for MC wakeup may be powered down.

Alternatively, power can be inductively coupled to the intra-vaginal device from an external source by means of an inductor and diode (not illustrated). External power may serve only to recharge battery 43. However, it may also be the intra-vaginal device's sole source of power, so that the device is active only when it receives external power. Then, battery 43 and power management circuit 45 may be dispensed with. However, the external power source must be kept near the monitored subject.

Device Sensors

Most embodiments of this invention include devices with an electrically-active sensor for detecting the occurrences of sexual activity, and the preferred sexual-activity sensor is MEMS-based accelerometer 49. A preferred accelerometer can measure positive and negative accelerations along at least a single axis, and preferably along two or more axes, the measured accelerations having amplitudes of approximately 1-5 g (acceleration of gravity), and frequencies from 0 Hz (static acceleration) to approximately 500 Hz and greater. Suitable low-power, accelerometers with on-chip signal conditioning circuitry are routinely available in packages down to 5 mm or less in all dimensions from, for example, Analog Devices, Inc. More preferred capacitive accelerometers, available from Tronics Microsystems SA, have very low power consumption but require off chip detection capabilities.

As FIG. 2 illustrates, accelerometer output is preferably linked to input 51 of a MC A/D converter without intervening parts. If accelerometer DC shift exceeds the ranges of the MC A/D, a sample and hold op-amp under MC control can offset accelerometer output before presentation to the A/D. The offset can be set as part of an initial calibration. Accelerometer 49, like optional sensors 53 and 55, is powered down by power control 45 when not in use. Accelerometers can be separately mounted in housing 1, as illustrated in FIG. 1, or can be mounted on flexible PCB 11.

A device may have other optional electrically-active sensors, for example, sensors 53 that are routinely available off-the-shelf, and sensors 55 that are specifically adapted to the preferred intra-vaginal device embodiment. Routinely available sensors 53 can include a temperature sensor (either a transistor junction or a thermistor), a pH sensor, sensors for particular compounds (for example, glucose), and the like. Suitable thermistors are available from Murata Manufacturing Co, Ltd., for example, model NCP15WD683J03RC having a maximum resistance of approximately 68 Kohms and a size of 0.12 mm by 0.06 mm (0402). Chemical sensors may detect electrical changes resulting from, for example, an enzymatic reaction of the substance to be detected, or from the absorption of a substance onto an active surface, and the like. Like accelerometer 49, optional sensors 53 are also powered through power control 45 and preferably provide their output signals directly to MC input.

Optional proximity/material sensors 55, on the other hand, are designed for the preferred intra-vaginal device and detect intra-vaginal pharmaceuticals, such as microbicides, pharmaceutical applicators, condoms, and the like (generally referred to here as “targets”). In one embodiment, sensors 55 are sensitive to field produced by a label, for example, label 67″, present in a target pharmaceutical, for example, pharmaceutical preparation 67′, or on a target applicator, or the like, instead of detecting the target directly. A preferred label produces static magnetic fields and is therefore not likely to otherwise be present in the environment or on other potential targets. Pharmaceutical and similar preparations can be suitably labeled with magnetic particles, such as magnetic micro-beads that are widely available both as off-the-shelf compositions and also as compositions tailored to particular specifications. Preferred beads are no larger than a few micrometers, produce a permanent magnetic field (alternately, have a large magnetic susceptibility), and have passivating surface treatments that promote dispersion and limit clumping. Suitable magnetic sensor ICs based on, for example, the Hall effect or on the giant magneto-resistive effect, are available off-the-shelf from, for example, Allegro Microsystems, Inc., Texas Instruments, Inc., Philips Semiconductors (Philips N.V.), and others. Physical objects, such as pharmaceutical applicators, condoms, and the like, can also be suitably labeled with magnetic material, configured, for example, as thin layer preferably no more than approximately 100 micrometers thick. Alternatively, a target label can be electrically active, being conductive, such as a metallic thin film, or having a permanent electric dipole moment, such as a ferroelectric composition, or otherwise. Electrically active labels are preferably detected by their capacitive effects on, e.g., a conductive configuration placed near the device environment at the surface the housing.

In another embodiment preferred for detecting physical objects, sensors 55 actively produce an electromagnetic field and detect its perturbation due to changes in the electromagnetic properties of the device environment caused by proximity of a target object. One such sensor includes an RF oscillator coupled to its environment through “antenna” 57 so that environmental changes, for example, the presence of applicator 61, interact with the antenna and alter the frequency or otherwise perturb the oscillator. For example, antenna 57 can be part of the frequency determining elements of the oscillator, so that induced changes in its inductive, capacitive, or resistive properties of the antenna perturbs oscillator frequency. The frequency can be perturbed by either “detuning” away from a normal frequency or by “un-detuning” back to a normal frequency; either change is then detected and provided to an MC input. The target object can be labeled to increase induced changes. In this embodiment, sensor 55 includes RF parts and/or frequency detection parts. Suitable RF oscillator/RF detector parts, either integrated or separately packaged, are available as part no. CMUT5159 in an SOT-523 package from. Suitable comparators (or combined RF detector and comparator) are available from National Semiconductor, Inc. as part nos. LMV331, LMV7235/7239.

Cooperating with active sensor 55, a target object, such as an applicator for an intra-vaginal pharmaceutical, can be provided with a capacitive element, for example, a conductive surface, or an inductive element, for example, a conductive loop, (a label) suitable for perturbing oscillator frequency by inducing 59 changes in antenna 57 properties. More preferably, the perturbing element is activated or inactivated during object use. For example, a simple switch, a membrane switch or similar, can be arranged so that, upon closing (or opening), it activates (or inactivates) the perturbing element in an applicator tip and “detunes” (or “un-detunes”) antenna 57 and RF oscillator 55, thus producing a unique and easily detected frequency signature. The membrane switch can be further configured and arranged so that it is necessarily opened or closed when the applicator is used. The invention includes the use of such labels in pharmaceutical preparations, as well as the use of other similarly-functioning types of labels.

Many embodiments of this invention also include sampling sensors that passively sample and bind, for example, pharmaceuticals, spermicides, microbicides, biologicals, biological macromolecules, biological agents, specific viruses, and so forth, that may be encountered in the device environment. Upon device removal, the sensors are destructively analyzed to determine what has been encountered and absorbed. Although sampling sensors are not usually electrically-active, in certain embodiments, these sensors may include electrically-active components, for example, an electrically active surface having properties dependent on adsorbed substances. Device can have two or more sampling sensors, for example, one directed to binding pharmaceuticals and another directed to binding viruses.

FIG. 1 illustrates generally sampling sensor 19. Sensor 19 resides in cavity 23 in ring housing 1. The cavity communicates to the external environment through port, pore, or array or ports or pores 21, and preferably, a membrane of controlled permeability (not illustrated). Membrane pore size, surface properties, and other characteristics are selected to preferentially admit the target components of interest. For example, the membrane may preferentially admit targets smaller than a selected pore size, which may range from approximately 30 nanometers or less to approximately 20 micrometers or greater; or it may preferentially admit targets with certain surface properties by providing membrane pores with hydrophilic, or hydrophobic, or charged surfaces; and the like. Suitable membrane materials are known in the art and include expanded poly-tetra-fluoro-ethylene, poly-ether-sulfone, and the like. Preferably adjacent to the external membrane is a wicking material that serves to efficiently transfer what the membrane has admitted to absorbent materials in the sensor cavity. Suitable wicking materials include precision-woven fabrics and/or porous ultra-high-molecular-weight-polyethylene (with optional surface treatments and/or surfactants).

Absorbent materials in the housing cavity bind and retain targets for later analysis. The surface characteristics of an absorbent material can be selected to bind a wide range of targets with little or no specificity or selectivity. In other embodiments, the surface characteristics can be selected to bind targets with various degrees specificity and selectivity. For example, the material may be generally hydrophilic or hydrophobic to generally bind only hydrophilic or hydrophobic targets. Or the material may have a positive, neutral, or negative surface charge to generally bind, for example, proteins, sugars, nucleic acids. In further embodiments, the absorbent material surface is selected to bind specific targets with particular selectivity. For example, the material may be primed to cross-link to certain classes of target. Further, the material may have surface antibody moieties specific to particular targets. Thereby, the absorbent material (along with the membrane) may be selected to bind specific biological agents such as the HIV virus, the Hepatitis B and/or C viruses, and similar.

The absorbent material's physical characteristics, for example, its diffusivity with binding affinity, can also be advantageously selected to provide information in addition to total exposure. By balancing a target's relative diffusivity and binding affinity, the spatial distribution of the target in the absorbent material can provide information on the time variability or lack thereof of the target's concentration in the device environment.

Contents accumulated in a sampling filter during residency in a participant are generally determined by destructive analysis using routine chemical and biological analytical techniques known in the art. For example, particular chemical compounds can be determined by chromatography, or by mass spectrometry, or the like. Particular complex molecules, such as proteins, nucleic acids, and biological agents containing such molecules, can be determined by routine immunologic assay (using specific antibodies, and the like). Biological agents can also be determined by culture techniques.

Methods Performed by the Device Micro-Controller

The device micro-controller (MC) reads data from electrically-active sensors, stores data in device memory, and manages overall device operations under the control of computer instructions (also referred to as “embedded software”) preferably residing in program memory integrated with the MC. Preferred, but non-limiting, embodiments of the embedded software and its memory structures are now described

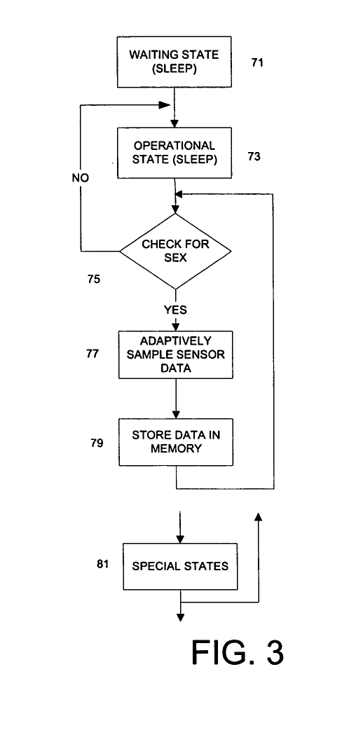

During its useful life, a device is advantageously described, and is described in the following, as progressing through a series of exemplary device states implemented by the embedded software. FIG. 3 illustrates that, from manufacture, or final assembly, or the like, until an insertion (or other) signal indicating use by a participant, the device is in wait state 71. In the wait state, the device merely waits for a use signal, consuming as little power as possible. Alternatively, if device power is connected only just prior to use, the initial wait state may be dispensed with. From device use or insertion until removal, that is when the device worn by a participant, the device is in operational state 73, during which it is fully functional for retrieving and storing sensor data. Typically, the embedded software also implements one or more special states 81. For example, a device can enter an end state when it detects insufficient power or removal from the participant, or the like. In the end state, the device configures itself to prevent data stored in memory from being corrupted. However, if on-board device data memory is read-only or otherwise configured to prevent corruption, the end data may be dispensed with. If a device is designed to be recharged or to transmit accumulated data while being worn by a participant, the embedded software advantageously implements special states to manage these activities. Further, states may be added as needed in specific embodiments; and details of processing within the states may be rearranged.

In more detail, wait state 71 is a special power-conserving state entered promptly after device battery power is first connected during initial manufacture, final assembly, or the like, but before use by a participant. In the wait state, the MC is in its lowest power sleep state after having executed a SLEEP instruction. (For ease of description, it is assumed herein that the MC enters a sleep state after executing a SLEEP instruction; the SLEEP instruction completes and the MC exits the sleep state in response to an enabled interrupt, either an external interrupt of an interrupt generated by an MC timer.) The MC wakes up in response to an external interrupt generated by a wake-up circuit. Only the MC and a wake-up circuit need be powered; all other device components can be powered down by power control 45 (FIG. 2). Preferably, the wake-up circuit includes a switch configured to be necessarily activated by flexing or deforming the device during insertion so that the wake up interrupt is automatically generated. For example, flexing or deforming during insertion can cause two closely opposed metal contacts within the housing to make mutual contact. Alternatively, the switch can include two external pads that are bridged during insertion by, for example, being touched by the subject. Generally, a device never need return to the wait state.

After exiting wait state 71 (or initially, if a wait state is not implemented), the device enters operational state 73 during which sensor data monitoring sexual activity is retrieved and stored. Because sexual activity is intermittent, power and memory can be advantageously further conserved, and device life further extended, by only intermittently sampling 75 for sexual activity, and by storing sensor detail data only if sexual activity is observed. If sexual activity is not detected, the device remains in a low-power sleep state. For example, the device may periodically wake up and sample for sexual activity approximately every 5 min., or preferably approximately every 5-10 min., or more preferably approximately every 10-20 min. or longer. The time spent in the sexual-activity check is preferably sufficient for necessary accuracy, but such that the overall device duty cycle is less than approximately 20%, preferably less than approximately 10%, and more preferably less than approximately 5% or less.

Preferably, sexual-activity check 75 extracts characteristics from an observed accelerometer signal, compares observed characteristics to a template indicating ranges of characteristics likely to indicate sexual activity, and indicates sexual activity is likely if the observed characteristics match the template. In one preferred embodiment, accelerometer signal characteristics extracted include the values of significant peaks in the accelerometer signal, representing significant acceleration of a device wearer, and the times of these peaks or the time intervals between these peaks. The template then describes one or more joint ranges of peak values and time intervals that have been determined as likely indicative of sexual activity. Additional discrminant characteristics can be advantageously used in further embodiments to increase accuracy of the sexual activity check. They include, for example, the regularity of the acceleration peaks, measured for example by standard deviations (or other statistical measure) of the peak values and/or time intervals between peaks; detection of recumbent posture as indicated by the direction of static acceleration (if measured by accelerometer 49); normal or above intra-vaginal temperature indicating increased local blood flow, and the like. Other discriminant characteristics may be available in various embodiments having various optional sensors.

Specific parameters of intermittent sampling and specific template ranges indicative of sexual activity are preferably determined by clinical trial and experiment. More preferably, they are customized for at least for each clinical trial, since the characteristics of sexual activity can vary from culture to culture, from region to region, and from individual to individual. However, this invention does not require 100% accuracy in checking for sexual activity. Accuracy need only be sufficiently for the intended statistical uses of device monitoring data in each clinical trial. Preferably, accuracy should be sufficiently so that trial conclusions may be reached more quickly and with a reduced number of participants.

With further reference to FIG. 3, in operational state 73 but when the device is neither sampling for sexual activity nor storing monitoring data, it enters a low-power sleep state. Prior to entering this sleep state, the MC controls power control 45 to power down external components not necessary for its subsequent wake-up, loads the sampling interval into an MC timer, and then executes a SLEEP instruction. When the timer interval expires, the SLEEP instruction completes, and the device again checks for sexual activity 75. If this check fails, the device again enters the sleep state. If it succeeds, the device proceeds to repetitively retrieve sensor data 77 and store retrieved data in memory 79. Preferably, intermittently during retrieved and storing data, the device checks 75 that sexual activity is continuing 75. If not, it powers down, but if so, it retrieved and storing. The device can also wake up from sleep state 73 upon the occurrence of selected external events. For example, the proximity/material sensor 55 may remain powered on, and generate an external wake up interrupt if it detects and pharmaceutical or a pharmaceutical applicator.

Data retrieval/sampling/storage, in less preferred embodiments, is performed at constant rates chosen in view of the Nyquist condition to provide sufficient signal bandwidth. Typical accelerometer sampling rates are approximately at least 10 Hz, or preferably approximately 20 Hz, to approximately 50 Hz or higher. Other sensors may be sampled less frequently, for example, for approximately 0.1 Hz to approximately 1 Hz. Digitization may be 8 bit or 16 bit.

However, in preferred embodiments, data is sampled at an adaptively set rate 77 to further conserve power and memory. Memory is further conserved by efficient data compression 79, and the preferred compression technique is selected to simplify and integrate with adaptive sampling. Briefly, for accelerometer data, only amplitudes of positive and negative signal peaks and the times of their occurrences (or time intervals between their occurrences, or their frequency) and extracted from the sampled accelerometer sensor data and stored in memory. This (lossy) compressed data are sufficient to determine and verify the sexual activity and also to adequately reconstruct the raw accelerometer signal if needed. Further, this data is adequately extracted from accelerometer signals that have been adaptively sampled so that a normal sampling rate and a normal MC clock rate are used for times near expected accelerometer signal peaks, while a lower sampling rate and a lower MC clock rate are used for times between expected accelerometer signal peaks. Such adaptive sampling 77 can advantageously reduce MC power consumption especially if the MC provides two or more selectable clock rates: a normal clock rate with normal power consumption for normal processing, and a slower clock rate with lower power consumption for slower processing. (If the chosen MC cannot be so controlled, adaptive sampling has less value.)

Adaptive sampling, accordingly, needs to be able to predict the approximate times of accelerometer signal peaks, and acquires parameters for this prediction are acquired during a brief initialization period of constant rate sampling (for example, at 10 Hz, or at 20 Hz, or at 50 Hz) at the commencement of device activity after device wake up. Initial initialization continues for a time sufficient to obtain a stable estimate of the time interval between significant acceleration signal peaks (or the equivalent). After initialization, adaptive sampling commences using normal (Nyquist) sampling and a normal MC clock in order to more accurately determine peak value/time pairs but with a reduced sampling rate and a slower MC clock between expected signal peaks. The duty cycle of the normal clock is preferably no more than approximately 50%, or preferably no more than approximately 25% or less. During reduces sampling, the MC looks for unexpected accelerometer signal peaks. If unexpected peaks occur (and optionally, intermittently), re-initialization is performed.

Other sensors optionally present in devices, for example, thermistors proximity/material sensors 55, and the like, generally produce data at much lower rates than does the accelerometer. Therefore, these sensors can be adequately sampled sampling only every second, or every 5 seconds, or every 10 seconds or longer. For example, these sensors can be sampled every 5^{th}, or every 10^{th}, or every 20^{th}, or every 50^{th} interval of normal clock processing during adaptive sampling. Certain sensors, for example, a proximity/material sensor and the like, detect events that can be signaled by interrupts and are preferably sampled when their interrupts are recognized.

Finally, the embedded software may provide special handling of the exceptional states described above. In the case of battery depletion, low voltage, and the like, the MC can shut down (HALT) to protect already stored data. If a device can be recharged or can communicate externally, when such a request is received, the MC performs the necessary hardware configuration actions, perhaps suspending any ongoing monitoring.

Sensed data is usually stored in the on-board memory as a sequential file, similar to a data log file, to which new data records are appended when available. Other suitable formats are known in the art. Data stored includes: sensor data, for example, accelerometer data, thermistor data, and so forth; time value data which indicate a current time; preferably, device data; and the like. Device data indicates, for example, device assembly time, wake up time from the initial wait state, wake time for checking sexual activity, whether or not sexual is detected, and the like. Various data encoding (for example, Huffman encoding) can be used for the actual data fields.

Data records have two preferred formats with other suitable formats being know in the art. Record Format 1 is a first preferred format.

RECORD FORMAT 1

| | | |
|---|---|---|
| DATA TYPE ID | VALUE | SEPARATOR |

Each format 1 record has a field for a single data value, a field whose value identifies the type of the data value, and a delimiter character, such as an ASCII comma or tab character, that separates records in the file. Time value records are regularly appended to the file, each time value record indicating the time that its subsequent data value records (or record) were sampled.

RECORD FORMAT 2

| | | | | |
|---|---|---|---|---|
| LEN | TIME VALUE | DATA TYPE ID1 | VALUE 1 | . . . |

Each format 2 record has all data values that were sensed at the time identified by the time value field. The first field is a length field of perhaps 4-8 bits (so no delimiter character is needed). Following the length field is the time value at which the subsequent data values were sampled. The record ends with one or more repeating groups of a data type identifier and an associated data value.

Methods Performed by an External Station

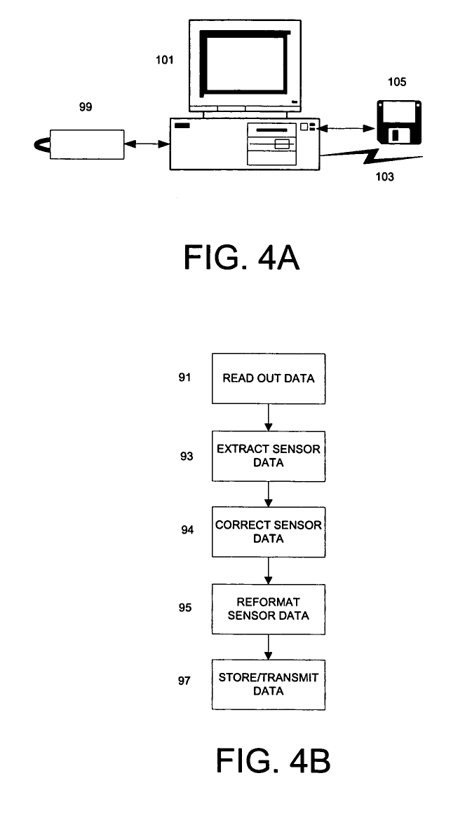

In most embodiments, data is retrieved from a device after its removal from a participant; in certain embodiments, data may be read out wirelessly with the device remaining in place. Once externally available, data is prepared and transmitted for ultimate use by a data station of this invention. FIG. 4A illustrates an exemplary station for data read out and preparation. Device reader 99 is adapted to contact the device's external contact pads (pads 17 in FIG. 1) and read out data 91 from the on-board memory. Retrieved data is transmitted across a standard interface to standard PC type computer 1101, where it is prepared for use and then transmitted by network 103 or by computer readable medium 105 to its ultimate user, for example, as part of a clinical trial. Additionally, the external station may sever to load or customize the embedded software, recharge the device, customize or calibrate the device and embedded software, and the like, prior to a device's use by a participant.

Generally, as much sensor data processing as possible is deferred to the external station. in order to conserve device resources and power. With reference to FIG. 4B, off-line processing of retrieved data begins with data extraction 93, which reads and interprets the device memory file, extracting and segregating data from each sensor, and time-stamping it with time values in the memory file. Next, errors, noise, and artifact apparent in sensor data are corrected 94. This step is unique to each sensor. For accelerometer data, DC or low frequency drift is recognized from a trend of peak signal values and is preferably removed by subtraction to recreate a substantially fixed “zero level”. Then, the measured peak values and time intervals are interpolated to obtain an estimated accelerometer waveform, zero-crossing times, frequency spectrum, and the like. Thermistor data correction 94 may involve no more than converting the measured data values to actual temperatures according to thermistor type. Finally, the corrected data is reformatted 95 into formats useful to the end users, and then forwarded 97 to these users. In other embodiments of off-line processing, the extraction, correction, and reformatting functions can be rearranged and repartitioned as apparent to one of average skill in the art.

Off-line processing also preferably extracts, summarizes and reports any device data present in the memory file. If sufficient device data has been logged, device reports can, for example, summarized the life cycle of a device, its manufacture, use, removal, any special events, and the like. Such reports can confirm the device was actually used as the participant claimed. Further, device reports can provide an audit trail of device processing, times of wakeup, results of sexual activity checks, and the like. Such reports can provide information for confirming device accuracy and/or adjusting software parameters.

The methods described here as executed in a device and in an external station in may be programmed in any convenient computer language, such as assembly language, C, or C++, compiled into an executable form and stored on a computer readable medium for loading into the device's program memory or into the off-line PC computer. However, for embedded software, a version of C adapted to for use with and to be stored in the particular MC is preferred. The present invention encompasses program products including computer readable media with the embedded software and/or the external station software.

### Additional Embodiments

The present invention also includes additional embodiments in which, for example, a intra-vaginal device of this invention is adapted for passive or active drug delivery. Passive intra-vaginal rings used for drug delivery are well known in the art. In certain passive drug-delivery rings, a pharmaceutical agent is mixed in bulk with silicone elastomer and molded into a ring in a manner so as to provide controlled release of the agent by diffusion from the elastomer. During use, the agent then controllably diffuses over a planned period of time from the elastomer ring into the vagina. In other passive drug-delivery rings, the pharmaceutical agent is concentrated in a cylindrical core, often only 2 mm in diameter and 5 mm in length, which is encased in a silicone elastomer sheath providing controlled diffusion of the agent from the core. Other designs are known in the art. See, for example, the Rathbone et al text cited above.

Intra-vaginal ring devices of this invention can readily be combined with either of these types of passive drug-delivery technology. In a first embodiment, the silicone elastomer from which the device is molded is selected also for controlled drug release and a pharmaceutical agent is mixed with the elastomer. The ring device in then molded from the elastomer-agent composite and will then additionally provide controlled drug release during its use. In a second embodiment, a drug-containing core is placed in a portion of the ring not occupied by electronics, sensors, or other internal components.

Active intra-vaginal drug-delivery rings are also known in the art. In certain embodiments, such active intra-vaginal ring devices include a chamber for storing a pharmaceutical agent, a gas chamber for storing pressurized gas that pressurizes and thereby causes release of the stored agent, and a subminiature solenoid valve (or similar device) for controlling the pressurized gas. See, for example, U.S. Pat. No. 5,928,195.

When these active drug delivery components (or similar) are combined with a device of this invention, the subminiature solenoid valve (or other control mechanism) can be activated by the device micro-controller upon detection of a predetermined condition. For example, drug delivery may be triggered when sexual activity is detected as described previously. In this event, released drugs can include microbicides, spermicides, hormones, and the like. For another example in women with a history of miscarriages, a progesterone-like agent can be released upon detection of a sustained rise in temperature, which is usually indicative of ovulation. Preferably, the log file accumulated by device of this invention would also include occurrences of the drug-delivery condition, occurrences of the initiation and/or termination of drug delivery, amount of drug remaining, and the like.

Other configurations of the device of this invention optionally designed for other body cavities can also be similarly provided with drug delivery capabilities.

### 6. EXAMPLE

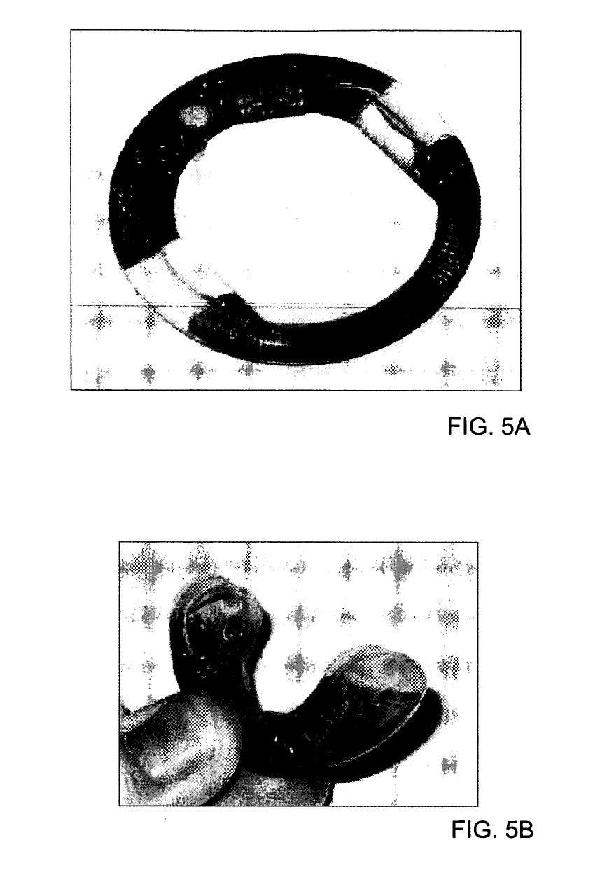

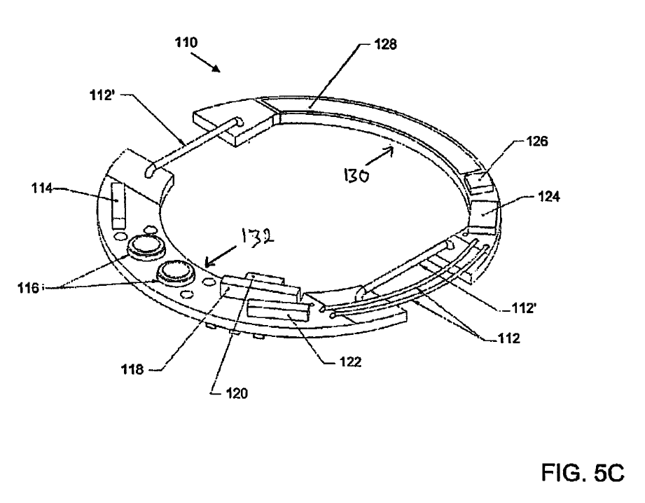

An example of an embodiment of the apparatus of this invention has been constructed. FIG. 5A generally illustrates that this example's housing is constructed from a substantially clear silicone elastomer and that is sized and shaped to reside in a subject's vagina. Generally, this example includes an accelerometer sensor and a transmitter that transmits accelerometer data externally to the subject.

FIG. 5B illustrates this example's flexibility, which is sufficient for routine insertion and removal by the subject.

FIG. 5C illustrates in more detail this example's internal construction 110. Components are mounted on two semicircular printed circuit boards (PCB) 130 and 132 that are linked by flexible power and signal wires 112 and grounding wires 112′. This split construction using two (or more than two) flexibly linked PCBs provides for the considerable flexibility illustrated in FIG. 5B even though the individual circuit boards may be less flexible.

The internal components mounted on the two PCBs include batteries 116. Switches 114 and 122 control the periods during which this example is powered for data collection and transmission. Vertical adapter PCB 118 mounts accelerometer 120 (Analog Devices, Inc.). Vertical adapter PCBs are useful to more compactly mount components with larger vertical dimensions. Accelerometer data is externally transmitted by RF transmitter 126 (Melexis Microelectronic Systems) packaged in a small outline package or smaller. Transmitter frequency is controlled by unit 124, which in this example is a crystal. Antenna 128 transmits externally the RF signal from transmitter 126.

It should be understood that this example apparatus exemplifies the inventions described above, and its particular features are not to be taken as limiting these inventions.

The invention described and claimed herein is not to be limited in scope by the preferred embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

A number of references are cited herein, the entire disclosures of which are incorporated herein, in their entirety, by reference for all purposes. Further, none of these references, regardless of how characterized above, is admitted as prior to the invention of the subject matter claimed herein.

What is claimed is:

1. An apparatus for monitoring a subject comprising:
a housing adapted to reside in an anatomic cavity of the subject's body, the housing having no external physical connection, and
at least one sensor of acceleration within the housing.

2. The apparatus of claim 1 wherein the anatomic cavity normally opens externally.

3. The apparatus of claim 2 wherein the housing is further adapted to permit insertion and removal by the subject.

4. The apparatus of claim 2 wherein the anatomic cavity is a vagina.

5. The apparatus of claim 4 wherein the housing is ring-shaped.

6. The apparatus of claim 1 further comprising at least one sensor selected from the group consisting of a sensor of pH, a sensor of temperature, a sensor of the occurrence of menstruation, a sensor of the proximity of magnetic materials, a sensor of the proximity of a target object.

7. The apparatus of claim 1 further comprising:
a computer-readable memory within the housing, and
a controller within the housing that retrieves measurements from at least one sensor and stores the retrieved measurements in the memory.

8. The apparatus of claim 1 further comprising:
an external unit, and
an RF transmitter within the housing that transmits sensor measurements to the external unit.

9. The apparatus of claim 1 further comprising a sampling sensor that samples components present in the external environment of the apparatus, the sampling sensor comprising:
an absorbent material disposed in a cavity in the housing, and
one or more pores through which components of the external environment have access to the absorbent material, wherein components are sampled by being absorbed by the absorbent material after passing though the pores from the external environment.

10. The apparatus of claim 1 further comprising a biosensor for sensing biological components, the biosensor being disposed in the housing and having access to biological components present in the external environment.

11. The apparatus of claim 10 wherein the biosensor comprises antibodies selective for one or more biological components.

12. The apparatus of claim 11 wherein the antibodies are selective for one or more sexually transmitted disease agents.

13. The apparatus of claim 12 wherein sexually transmitted disease agents comprise the human immunodeficiency virus (HIV).

14. An apparatus for monitoring a female subject comprising:
a housing adapted to reside in the subject's vagina, the housing having no external physical connection, and
one or more sensors within the housing comprising at least one sensor selected from the group consisting of a sensor of acceleration, a sensor of pH, a sensor of temperature, a sensor of the occurrence of menstruation, a sensor of the proximity of magnetic materials, and a sensor of the proximity of a target object.

15. The apparatus of claim 14 wherein the housing is further adapted to permit insertion and removal by the subject.

16. The apparatus of claim 14 wherein the housing is ring-shaped.

17. The apparatus of claim 14 wherein at least one sensor is selected from the first group consisting of a sensor of pH and a sensor of temperature,
and at least one other sensor selected is from second the group consisting of a sensor of acceleration, a sensor of the occurrence of menstruation, a sensor of the proximity of magnetic materials, a sensor of the proximity of a target object.

18. The apparatus of claim 14 further comprising:
a computer-readable memory within the housing, and
a controller within the housing that retrieves measurements from at least one sensor and stores the retrieved measurements in the memory.

19. The apparatus of claim 18 comprising a sensor of acceleration, and wherein acceleration measurements stored in the memory by the controller comprise peak values of the measured acceleration and the occurrence times of the measured peak values.

20. The apparatus of claim 19 wherein the controller determines whether or not the subject is determined as likely to be engaging in sexual activity, or as not likely to be engaging in sexual activity, by comparing characteristics of the acceleration peak values and of the peak-value occurrence times with characteristics expected if the subject is actually engaging in a sexual activity.

21. The apparatus of claim 14 further comprising:
an external unit, and
an RF transmitter within the housing that transmits sensor measurements to the external unit.

22. The apparatus of claim 14 further comprising a sampling sensor that samples components present in the external environment of the apparatus, the sampling sensor comprising:
an absorbent material disposed in a cavity in the housing, and
one or more pores through which components of the external environment have access to the absorbent material, wherein components are sampled by being absorbed by the absorbent material after passing though the pores from the external environment.

23. The apparatus of claim 22 wherein the sample components comprise one or more sexually transmitted disease agents.

24. The apparatus of claim 14 further comprising a biosensor for sensing biological components, the biosensor being disposed in the housing and having access to biological components present in the external environment.

25. The apparatus of claim 24 wherein the biosensor comprises antibodies selective for one or more biological components.

26. The apparatus of claim 15 wherein the antibodies are selective for one or more sexually transmitted disease agents.

27. The apparatus of claim 26 wherein sexually transmitted disease agents comprise the human immunodeficiency virus (HIV).

28. An apparatus for monitoring a subject comprising:
a housing adapted to reside in an anatomic cavity of the subject's body, the housing having no external physical connection, and
at least one sensor of the proximity of magnetic materials within the housing.

29. The apparatus of claim 28 further comprising a pharmaceutical preparation applied in the proximity of the apparatus within the anatomic cavity, the pharmaceutical preparation comprising magnetic materials.

30. The apparatus of claim 28 further comprising one or more sensors of acceleration.

31. The apparatus of claim 28 further comprising:
a computer-readable memory within the housing, and
a controller within the housing that retrieves measurements from at least one sensor and stores the retrieved measurements in the memory.

32. The apparatus of claim 28 further comprising:
an external unit, and
an RF transmitter within the housing that transmits sensor measurements to the external unit.

33. An apparatus for monitoring a subject comprising:
a housing adapted to reside in an anatomic cavity of the subject's body, the housing having no external physical connection, and
at least one sensor of the proximity of a target object within the housing.

34. The apparatus of claim 33 wherein the proximity sensor generates an electromagnetic field and senses perturbations in the electromagnetic field, and
wherein the target object perturbs an electromagnetic field and is thereby detected.

35. The apparatus of claim 34 wherein the target object comprises a conductive element and/or an inductive element that perturbs an electromagnetic field.

36. The apparatus of claim 33 wherein the target object comprises an applicator for a pharmaceutical.

37. The apparatus of claim 33 wherein the target object comprises a contraceptive device.

38. The apparatus of claim 37 wherein the contraceptive device comprises a condom.

39. The apparatus of claim 33 further comprising one or more sensors of acceleration.

40. The apparatus of claim 33 further comprising:
a computer-readable memory within the housing, and
a controller within the housing that retrieves measurements from at least one sensor and stores the retrieved measurements in the memory.

41. The apparatus of claim 33 further comprising:
an external unit, and
an RF transmitter within the housing that transmits sensor measurements to the external unit.

42. An apparatus for monitoring a subject comprising:
a housing adapted to reside in an anatomic cavity of the subject's body, the housing having no external physical connection, and
a sampling sensor for sampling components of the external environment of the apparatus, the sampling sensor comprising:
an absorbent material disposed in a cavity in the housing, and
one or more pores through which components of the external environment have access to the absorbent material, wherein components are sampled by being absorbed by the absorbent material after passing though the pores from the external environment.

43. The apparatus of claim 42 wherein the anatomic cavity is a vagina.

44. The apparatus of claim 42 wherein adsorbent material and/or the pores are selected so that the pre-determined components of the external environment are preferentially sampled.

45. The apparatus of claim 42 wherein the external components sampled comprise biological agents and/or biological molecules.

46. The apparatus of claim 45 wherein the biological agents comprise one or more sexually transmitted disease agents.

47. The apparatus of claim 46 wherein sexually transmitted disease agents comprise the human immunodeficiency virus (HIV).

48. The apparatus of claim 42 wherein the absorbent material comprises antibodies selective for one or more biological components.

49. The apparatus of claim 48 wherein the antibodies are selective for one or more sexually transmitted disease agents.

50. The apparatus of claim 42 further comprising:
one or more sensors within the housing,
a computer-readable memory within the housing, and
a controller within the housing that retrieves measurements from at least one sensor and stores the retrieved measurements in the memory.

51. The apparatus of claim 42 further comprising:
one or more sensors within the housing,
an external unit, and
an RF transmitter within the housing that transmits sensor measurements to the external unit.

52. An apparatus for monitoring a female subject's sexual activity comprising:
a housing adapted to reside in the subject's vagina, the housing having no external physical connection,
one or more sensors within the housing,
a computer-readable memory within the housing,
a controller within the housing, and
a program for
awakening periodically from a low power sleep state in order to determine from sensor measurements whether or not the subject is likely to be engaging in sexual activity,
retrieving measurements from at least one sensor and storing the retrieved measurements in the memory during a period in which the subject is determined as likely to be engaging in sexual activity, and
switching back to the low power sleep state if the subject is determined as not likely to be engaging in sexual activity.

53. The apparatus of claim 52 wherein the one or more sensors comprise at least one sensor of acceleration, and
wherein measurements are retrieved from the acceleration sensor, and wherein the controller stores in the memory peak values of the measured acceleration and the occurrence times of the measured peak values.

54. The apparatus of claim 53 wherein the subject is determined as likely to be engaging in sexual activity, or as not likely to be engaging in sexual activity, by comparing characteristics of acceleration peak values and of peak-value occurrence times with characteristics expected if the subject is actually engaging in a sexual activity.

55. The apparatus of claim 53 wherein acceleration measurements are adaptively retrieved and stored so that the acceleration is more frequently sampled near the expected times of an acceleration peak value, and is less frequently sampled between the expected times of acceleration peak values.

56. The apparatus of claim 52 further comprising a sampling sensor for sampling components of the external environment of the apparatus, the sampling sensor comprising:
an absorbent material disposed in a cavity in the housing, and
one or more pores through which components external environment has access to the absorbent material, wherein components are sampled by being absorbed by the absorbent material after passing though the pores from the external environment.

57. The apparatus of claim 56 wherein the external components sampled comprise one or more sexually transmitted disease agents.

58. The apparatus of claim 52 further comprising at least one sensor selected from the group consisting of a sensor of pH, a sensor of temperature, a sensor of the occurrence of menstruation, a sensor of the proximity of magnetic materials, and a sensor of the proximity of a target object.

59. The apparatus of claim 52 further comprising a biosensor for sensing biological components, the biosensor being disposed in the housing and having access to biological components present in the external environment.

60. The apparatus of claim 59 wherein the biosensor comprises antibodies selective for one or more biological components.

61. The apparatus of claim 60 wherein the antibodies are selective for one or more sexually transmitted disease agents.

62. The apparatus of claim 61 wherein sexually transmitted disease agents comprise the human immunodeficiency virus (HIV).

63. An apparatus for subject monitoring and drug delivery comprising:
a housing adapted to reside in an anatomic cavity of the subject's body, the housing having no external physical connection; the housing comprising an embedded pharmaceutical agent that diffuses from the housing into the anatomic cavity, and
one or more sensors within the housing.

64. The apparatus of claim 63 wherein the pharmaceutical agent is embedded throughout the housing material.

65. The apparatus of claim 63 wherein the pharmaceutical agent is embedded in a core of the housing.

66. The apparatus of claim 63 wherein the sensors comprise one or more sensors of acceleration.

67. The apparatus of claim 63 wherein at least one sensor is selected from the first group consisting of a sensor of pH and a sensor of temperature,
and at least one other sensor selected is from second the group consisting of a sensor of acceleration, a sensor of the occurrence of menstruation, a sensor of the proximity of magnetic materials, a sensor of the proximity of a target object.

68. The apparatus of claim 63 further comprising:
a computer-readable memory within the housing, and
a controller within the housing that retrieves measurements from at least one sensor and stores the retrieved measurements in the memory.

69. The apparatus of claim 63 further comprising:
an external unit, and
an RF transmitter within the housing that transmits sensor measurements to the external unit.

70. An apparatus for subject monitoring and drug delivery comprising:
a housing adapted to reside in an anatomic cavity of the subject's body, the housing having no external physical connection,
a reservoir of a pharmaceutical agent within the housing, and
one or more sensors within the housing,
a controller within the housing, and
a program for
retrieving measurements from at least one sensor and determining whether or not a condition is satisfied in dependence on the retrieved measurements, and
if the condition is determined to be satisfied, signaling that an amount of the pharmaceutical agent is to be released in the anatomic cavity.

71. The apparatus of claim 70 wherein the condition to be satisfied comprises whether the subject is likely to be engaging in a sexual activity.

72. The apparatus of claim 70 wherein the one or more sensors is a sensor of acceleration, and
wherein the controller determines whether or not the subject is likely to be engaging in a sexual activity by comparing characteristics of acceleration peak values and of occurrence times of the acceleration peak values with characteristics expected if the subject is actually engaging in a sexual activity.

73. The apparatus of claim 70 further comprising:
a computer-readable memory within the housing, and
wherein the program further stores retrieved measurements in the memory.

74. The apparatus of claim 70 further comprising:
an external unit, and
an RF transmitter within the housing that transmits sensor measurements to the external unit.

75. A computer readable memory with a program for:
awakening periodically from a low power sleep state in order to determine from acceleration measurements whether or not the subject is likely to be engaging in sexual activity,
retrieving measurements from at least one sensor and storing the retrieved measurements in the memory during a period in which the subject is determined as likely to be engaging in sexual activity, and
switching back to the low power sleep state if the subject is determined as not likely to be engaging in sexual activity.

76. A system from monitoring female subjects comprising:
a monitoring apparatus comprising
a housing adapted to reside in the subject's vagina, the housing having no external physical connection, and
one or more sensors within the housing comprising at least one sensor selected from the group consisting of a sensor of acceleration, a sensor of pH, a sensor of temperature, a sensor of the occurrence of menstruation, a sensor of the proximity of magnetic materials, and a sensor of the proximity of a target object,
a computer-readable memory within the housing, and
a controller within the housing that retrieves measurements from at least one sensor and stores the retrieved measurements in the memory, and
a computer for reading measurements stored in the memory of the monitoring apparatus during a prior period of residence in the subject.

77. A method of conducting clinical trials using a plurality of subjects comprising:
providing to at least one subject a monitoring apparatus comprising
a housing adapted to reside in the subject's vagina, the housing having no external physical connection, and
one or more sensors within the housing comprising at least one sensor selected from the group consisting of a sensor of acceleration, a sensor of pH, a sensor of temperature, a sensor of the occurrence of menstruation, a sensor of the proximity of magnetic materials, and a sensor of the proximity of a target object,
a computer-readable memory within the housing, and
a controller within the housing that retrieves measurements from at least one sensor and stores the retrieved measurements in the memory,
reading measurements stored in the memory of the monitoring apparatus after a period of subject use, and
transmitting the measurements read for analysis.

78. A pharmaceutical preparation comprising:
one or more pharmaceutical agents; and
embedded magnetic materials sufficient to permit a sensor for magnetic materials to sense the proximity of the pharmaceutical preparation.

79. An apparatus for monitoring a female subject's sexual activity comprising:
a housing adapted to reside in the subject's vagina, the housing having no external physical connection,
one or more sensors including at least one sensor of acceleration within the housing,
a controller within the housing that determines whether or not the subject is likely to be engaging in sexual activity, or is not likely to be engaging in sexual activity, by comparing characteristics of the acceleration signals with characteristics expected if the subject is actually engaging in a sexual activity.

80. The apparatus of claim 79 further comprising at least one sensor selected from the group consisting of a sensor of pH, a sensor of temperature, a sensor of the occurrence of menstruation, a sensor of the proximity of magnetic materials, and a sensor of the proximity of a target object.

81. The apparatus of claim 79 further comprising a biosensor for sensing biological components, the biosensor being disposed in the housing and having access to biological components present in the external environment.

82. The apparatus of claim 59 wherein the biosensor comprises antibodies selective for one or more biological components.

83. The apparatus of claim 60 wherein the antibodies are selective for one or more sexually transmitted disease agents.

84. The apparatus of claim 61 wherein sexually transmitted disease agents comprise the human immunodeficiency virus (HIV).

85. The apparatus of claim 63 further comprising a computer-readable memory within the housing, and wherein the controller within the housing that retrieves measurements from at least one sensor and stores the retrieved measurements in the memory.

86. The apparatus of claim 63 further comprising:
an external unit, and
an RF transmitter within the housing that transmits sensor measurements to the external unit.
